(19) United States
(12) Patent Application Publication
(10) Pub. No.: US 2016/0158292 A1
(43) Pub. Date: Jun. 9, 2016

(54) METHOD AND APPARATUS FOR RECOVERY OF UMBILICAL CORD TISSUE DERIVED REGENERATIVE CELLS AND USES THEREOF

(71) Applicant: Ingeneron, Inc., Houston, TX (US)

(72) Inventors: Eckhard Alt, Houston, TX (US); Ivonne Guzman Bruno, Pearland, TX (US); Timo Nazari Shafti, Houston, TX (US); Michael E Coleman, Houston, TX (US); Anita Kadala, Houston, TX (US)

(73) Assignee: Ingeneron, Inc., Houston, TX (US)

(21) Appl. No.: 14/815,808

(22) Filed: Jul. 31, 2015

Related U.S. Application Data
(60) Provisional application No. 62/122,267, filed on Jul. 29, 2014.

(51) Int. Cl. A61K 35/50; A61K 9/00; C12Q 1/68

(52) U.S. Cl. CPC A61K 35/50; C12Q 1/6881; A61K 9/0019; C12Q 2600/158

(57) ABSTRACT

Methods for the extraction and processing of umbilical stem cells for therapeutic and diagnostic purposes are disclosed. The methods provide high regenerative cell numbers and high cell viability. Further, the methods do not require culturing the cells in serum or growth factors.

### CROSS-REFERENCE TO RELATED APPLICATIONS

This application is related to U.S. Provisional Application No. 62/122,267, filed Jul. 29, 2014, with the same inventors herein, as to which a claim of priority is made for common subject matter and which is incorporated herein by reference.

### FIELD OF THE INVENTION

The present invention generally relates to methods, systems and apparatuses for recovery of cells from umbilical cord and uses thereof. More specifically, the present invention relates to harvesting an umbilical cord, processing the umbilical cord to obtain umbilical cord tissue, processing that tissue to obtain a cell preparation, preserving the cell preparation so obtained, and using cells therefrom for therapeutic, preventive or diagnostic purposes.

### BACKGROUND OF THE INVENTION

In recent years, stem cells have received considerable attention from basic scientists and clinicians seeking to develop strategies for rebuilding tissues and restoring critical functions of diseased, aged, or damaged tissues. Stem cells hold significant promise for tissue repair and regeneration, prevention of further tissue damage, and for diagnostic purposes.

Stem cells have the ability to divide (self-replicate) theoretically indefinitely, even throughout the life of an organism. Under the right micro-environmental conditions, or given the right signals, stem cells can give rise to (“differentiate into”) the many different cell types that make up the organism. That is, stem cells have the potential to develop into differentiated cells that have characteristic shapes and specialized functions, such as heart muscle, skin cells, hepatic tissues, or nerve cells.

Stem cells are generally classified as adult stem cells, embryonic stem cells, or induced pluripotent adult stem cells with embryonic characteristics. Pluripotent cells are stem cells that can differentiate into all three embryonic germ layers (i.e., mesoderm, endoderm, and ectoderm).

Embryonic and adult stem cells differ from each other in that embryonic cells and induced pluripotent cells can form new tissue without guidance and independently from existing tissue whereas adult stem cells typically require guidance from existing (i.e., host) tissues. An embryo or fetus generally requires stem cells for organ development, that is, to create organs not yet existing by means of embryonic stem cells. And embryonic stem cells display strong proliferation and differentiation potential both in vitro and in vivo. Their use for therapeutic, preventive, diagnostic or research purposes, however, has been severely limited in view of the presence of issues including ethical concerns, potential immunological rejection and the risk of teratoma formation.

Accordingly, adult stem cells (often referred to as regenerative cells) have been identified and investigated in various organs and tissues^{1,2} [Note: Superscript numbers refer to References listed at the end of the Detailed Description]. These include virtually any acquired from tissue having vasculature, such as bone marrow, placenta, liver, et cetera, but among adult stem cells most preferred for therapeutic and research purposes in recent times are those derived from adipose tissue. Early stem cells are intra and perivascular; their frequency correlates with the blood vessel density and the stromal fraction. Of note, among resident regenerative cells found in adipose tissue are considerable numbers of pluripotent stem cells. As a result, adult (and for reasons noted above, not embryonic) stem cells have become increasingly important in developing innovative therapeutic strategies for overcoming tissue damage.

Unlike cancer cells, which can differentiate into different lineages without guidance from pre-existing structures, adult pluripotent stem cells only differentiate into tissues that already exist. Thus, while metastatic cancer cells take their own form in other organs that are not their primary cellular origin, adult pluripotent stem cells receive signaling from the new tissue microenvironment and are guided to differentiate into the cellular equivalent of the host tissue where they are placed or needed. This means if stem cells/regenerative cells are administered to and in contact or sufficiently close relation with a specific microenvironment such as tissue of the heart, liver, cartilage, or nerves, they acquire the characteristics and function, and differentiate into the existing cellular lineage dictated by that environment.

The ability of adult pluripotent stem cells to receive signaling from the new tissue microenvironment and to be guided to differentiate into that tissue is important in acting to preclude any differentiation into other tissues. Thus, cells that are obtained from any tissue containing blood vessels (i.e., vasculature, and thereby possibly pluripotent stem cells) such as subcutaneous fat tissue, for example, can only differentiate into new different tissue as determined and guided by the microenvironment of the new tissue.

Responsiveness to the microenvironment is important to prevent tumor formation as well as formation of tissue in an ectopic manner, such as formation of bone in a heart or adipose structure in a knee. But there remain certain limitations on use of autologous adult cells. With aging, cells replicate at a slower rate and their capacity to differentiate into certain lineages is diminished. Nature has provided this as a safety means to prevent the occurrence of cancer. While shortening of telomeres and reduction in telomerase activity contribute to this slowdown of differentiation capacity, other age-associated physiologic changes also diminish cellular capacity for differentiation. The magnitude of these changes varies between individuals, and, with increasing age, autologous stem cell therapy may have limited efficacy in some individuals.

The use of allogeneic cells from younger individuals may prove beneficial in alleviating some of the issues associated with damage, ageing or diseases, such as genetic diseases. That is, stem cells in these cases obtained from one individual are applied to another individual (i.e., allogeneic transfer). But this method requires performing an allogeneic match of cell surface markers to determine the cell surface immunotyping of the respective cells.

Some current stem cell therapy requires culturing the stem cells. Primarily, this is done to obtain a sufficient number of cells. For example, bone marrow-derived cells typically contain less than 0.1% true stem cells. Additionally, more than 99% of those bone marrow-derived cells are progenitor cells that are dedicated to a hematological differentiation when fully matured. It is therefore less beneficial to provide stem cell therapy with bone marrow-derived cells in a foreign environment that does not provide the clues of hematopoietic guidance by the microenvironment upon maturation. As a result, if other local microenvironmental clues do not confirm the presence of the “right” hematopoietic location, the stem/progenitor cells induce an apoptosis program to stop that differentiation and to destroy themselves. Without apoptosis, these cells would cause a major inflammation at the injection site since an increased and higher number of inflammatory cells would accumulate if those cells were allowed to continue to mature to hematopoietic lineages such as macrophages, CD4+ lymphocytes, neutrophilic, basophilic and/or eosinophilic cells of the immune system.

It would therefore be advantageous to provide methods, systems and apparatuses for recovery of pluripotent stem cells, or regenerative cells, in large numbers from an organ or tissue that is not critical to an ongoing important function in the body, such as from umbilical cord that is typically discarded after infant delivery. More specifically, the present invention relates to processing umbilical cord tissue, extracting the cells from that tissue, and using cells therefrom for various therapeutic, preventive and diagnostic purposes.

### SUMMARY OF THE INVENTION

Certain aspects of the present invention concern methods of processing tissue for use in regenerative medicine. In one such aspect, this includes 1) harvesting an umbilical cord from a donor; 2) extracting tissue comprising nucleated cells from the umbilical cord; 3) contacting the tissue with an enzymatic solution; 4) subjecting the tissue to agitation; 5) filtering the tissue after agitation; 6) subjecting the filtered tissue to centrifugation; 7) concentrating the tissue to produce a cell solution; 8) washing the cell solution; and thereupon recovering a cell preparation containing a population of regenerative cells including pluripotent stem cells.

In another aspect of the invention, the tissue is processed and the resulting cell preparation administered within one and the same medical procedure. In yet another aspect of the invention, the umbilical cord is processed at the site of delivery of the newborn umbilical cord donor from its mother. In a further aspect, the harvested umbilical cord is segmented and the segments of the umbilical cord are processed in the presence of a reagent, the processed segments are filtered to produce a cell solution, the cell solution is washed and centrifuged to recover a cell preparation or population, wherein the recovered cell preparation includes pluripotent stem cells.

In yet a further aspect of this invention, the resulting cell preparation is administered to the infant umbilical cord tissue donor to alleviate birth or delivery-related complications including, for example, cerebral hypoxia. In another aspect of this invention, the cell preparation contains a number (i.e., a sufficiently large population) of regenerative cells sufficient for therapeutic purposes without expansion. In another aspect, the cell preparation is used in a matched or unmatched allogeneic manner. For matching, the future human leukocyte antigen (HLA) immune markers of the as-yet nonexisting HLA markers (i.e., stem cells are initially immune-privileged but develop and express the HLA type of the donor after maturation) a nucleated cell sample of the donor is analyzed for determination of the HLA type of the donor cells. This information is used to predict the HLA type the stem cell preparation will assume upon maturation and also to match HLA markers. According to a further important aspect of this invention, the capability to identify and actual identification of HLA type or markers enables practicality of establishing a tissue or regenerative cell storage bank in which cryopreserved tissue/stem cells are stored according to their identified HLA type or markers, for subsequent use (typically, but not necessarily, allogenically) in therapeutic, preventive, diagnostic or research procedures.

The tissue extracted from the umbilical cord includes Wharton's jelly, blood vessels and stroma including connective tissue. In one embodiment or method of the invention, the extracted umbilical cord tissue is diced after the extraction, and prior to contacting the tissue with an enzymatic solution, which solution preferably comprises a collagenase and neutral protease blend, for example. The tissue is immersed in the enzymatic solution before and at time of processing.

Following contacting the tissue with the enzymatic solution (including soaking the umbilical cord tissue for a certain period of time), the tissue and enzymatic solution are subjected to processing and centrifugation. Short-term repetitive sequences of agitation are typically performed at a rate and length of time such that cells are not destroyed. And the processing of the cord tissue is typically performed at human body temperature. For non-human cells, such as cells of other mammals, the tissue can either be processed at the normal body temperature of the donor mammal or at the aforementioned human body temperature.

In certain aspects of the invention, after processing, a pellet and a supernatant are present. In these aspects, the supernatant is removed and subjected to membrane filtration to recover regenerative cells. The membrane filter may be any size amenable to filtering cells and preventing tissue larger than single cells from passing through the membrane. In certain instances this is accomplished with a 100 micron filter.

In an exemplary method of the types referred to above, results demonstrate a yield of between 8 and 20% regenerative cells. And such method typically results in cells having a viability of 85% or greater. Preferably, the umbilical cord is processed within a few hours after newborn delivery, at the site of delivery (point-of-care).

In some embodiments of the invention, the recovered regenerative cells represent stem cells and progenitor cells, wherein a certain population or subpopulation of the cells are pluripotent. Further, upon filtration and subsequent concentration, the cells can be administered to a subject in need of regenerative cell therapy, and in accordance with certain methods of the invention, administration of the regenerative cells may be performed without need for culturing prior thereto.

In cases where immediate administration to a subject in need of regenerative cell therapy is not prescribed, the recovered cells or cell populations may be subjected to another concentration and resuspended in a cryoprotectant, to be frozen and stored (banked). Later, these cryopreserved cells and populations, properly identifiable as described above and in the following detailed description, may be retrieved even years later from storage for use in medical procedures.

Therefore, it is a principal aim of the present invention to provide a system, technique or method for organization of banks of cryopreserved umbilical cord tissue-derived stem cells based on regional probability of typing, the publication of the available cells of the banks in a data registry so that a potential recipient can obtain its own HLA typing and immune cell surface characterization in order to find a perfect match. Information on typing of available cells may be made available via a remotely accessible electronic database.

A further aim of the present invention is to make use of the individual properties and specific benefits not only of umbilical cord tissue-derived cells, but also of umbilical cord blood-based cells from the same donor, and the additional benefits achieved when the two different cell types are applied together to a recipient, i.e., cord tissue-derived stem cells together with umbilical cord blood-derived stem cells.

An additional aim is to utilize the HLA type and markers of the cord blood-derived cells for purposes of identification of that of the cord tissue-derived cells of the same donor (the HLA type and markers of the latter not otherwise available at the outset when cells are to be cryopreserved and banked), which is essential for banking of large numbers of cells of different donors and different HLA types for subsequent allogeneic transfer.

It is to be emphasized and understood that discussion of any aspect of the invention may be applicable to other aspects of the invention as well. For example, the disclosures set forth in the Example section below refer to aspects applicable to all aspects of the invention.

Use of the term “or” in the claims means “and/or” unless explicitly indicated to refer to alternatives only or the alternatives are mutually exclusive, although the disclosure supports a definition that refers to only alternatives and “and/or.” Throughout this application, the term “about” is used to indicate that a value includes the standard deviation of error for the device or method being employed to determine the value. As used in this specification, “a” or “an” may mean one or more than one. As used in the claim(s) herein, when in conjunction with the word “comprising” or words of similar import, the words “a” or “an” may mean one or more than one. And as used herein, “another” may mean at least a second or more.

Other aims, objects, features and attendant advantages of the present invention beyond those referred to or inferred from the above, will become apparent from the following detailed description of presently preferred embodiments and methods of the invention. It should be understood, however, that the detailed description and the specific examples, while indicating aspects of the invention, are given by way of illustration only, since various changes and modifications within the spirit and scope of the invention will become apparent to those skilled in the art from the detailed description.

### BRIEF DESCRIPTION OF THE DRAWINGS

FIG. 1 is a schematic enlarged perspective representation of a section of an umbilical cord, illustrating the umbilical cord tissue (UCT) including vessels, surrounded by the tissue structure known as Wharton's jelly and stroma, wherein the Wharton's jelly encompasses the vascular and subamnionic regions and is walled by the amnion epithelium.

FIGS. 2A-I represent a photographic sequence of a dissection of an umbilical cord sample, illustrating in FIG. 2A, the umbilical cord anatomy; in FIG. 2B, removal of the umbilical cord amnion epithelium with a hemostat; in FIG. 2C and FIG. 2D, expansion of the umbilical cord vessels using medium size scissors to facilitate the separation of the vessel from the Wharton's jelly intervascular tissue; in FIG. 2E, FIG. 2F and FIG. 2G, stepwise dissection of the Wharton's jelly tissue from other structures such as the vessel walls is shown; in FIG. 2H, umbilical cord tissue including vessel, stroma and Wharton's jelly tissue is shown after dissection; and in FIG. 2I, the resulting umbilical cord tissue is shown.

FIG. 3 is a multi-step diagram illustrating steps of the processing of umbilical cord tissue including Wharton's jelly tissue using a semi-automated system, wherein in step 1, dissection of the umbilical cord tissue (UCT) and subsequent mincing of the UCT is performed; in step 2, processing buffer and MATRASE™ Reagent are added to the minced tissue and incubated for 1 to 4 hours in an InGeneron™ ARC processing unit at 37 to 40° C. under automated constant agitation; in step 3, supernatant of the processed tissue is passed through a 100 μm filter to remove debris and unprocessed tissue; in step 4, after filtration the resulting cell suspension is spun down and the cell pellet is washed two to three times to remove any remaining enzyme and tissue debris following the decomposition of the umbilical cord tissue with, for example, the MATRASE™ Reagent; and in a final step 5, the final cell pellet can be re-suspended in desired carrier solution and administered or cryopreserved.

FIG. 4A is a stepwise appearance of umbilical cord tissue and cells during processing and culture, showing 1, umbilical cord tissue including Wharton's jelly, vessels, and stroma after one hour of processing with MATRASE™ Reagent with automated mechanical agitation; 2, cell pellet appearance after the first filtration and concentration step; and 3, cell pellet appearance after washes, exhibiting umbilical cord regenerative cells (UC-RC). FIG. 4B is a table indicating total nucleated cell counts, percent viability, and percent CFU-F (i.e., colony forming unit microblast) of freshly isolated cells from umbilical cord tissue. FIG. 4C illustrates light microscopy of passage 0 (i.e., first culture following the isolation of cells from tissue; also called the primary culture) adherent cell fraction (umbilical cord content of perinatal, plastic adherent mesenchymal stem cells, or UC-MSC) from umbilical cord tissue after 4 days in culture.

FIG. 5 represents multiple images of a differentiation potential of umbilical cord tissue stem cells in culture. UC-MSCs obtained using the semi-automated protocol were cultured for 2-3 weeks under differentiation-inducing conditions. The Figure comprises light microscopy images of UC-MSC differentiated in chondrogenic (mesoderm), osteogenic, hepatogenic (endoderm), senescence, adipogenic, and neurogenic (ectoderm) media.

FIG. 6A is a comparison of gene MSC expression profiling of UC-MSCs isolated with this presently-preferred protocol (WJSCs, i.e., Wharton's jelly stem cells) compared to adipose tissue MSCs (ADSCs) and bone marrow MSCs (BMSCs). In FIG. 6B, a scatter plot comparison of the normalized expression of genes associated with stemness and mesenchymal characteristics (MSC array, SABioscience, Qiagen, Inc., Valencia, Calif.) UC-MSC (in this figure, designated USCs) expression levels (darker circles) were plotted against adipose derived stem cells (in this figure, designated ASCs, lighter circles), and in FIG. 6C, plotted against bone marrow-derived stem cells (BMSCs, lighter circles), to quickly visualize possible differences in gene expression. The central sloped line in FIGS. 6B and 6C indicates unchanged gene expression. Genes above the central lines indicated higher expression levels; whereas, genes below central lines indicate lower expression levels for UC-MSC.

FIG. 7 consists of sets of light microscopy images showing, in the set of the upper row, that the muscle cells of a patient with Duchenne disease (Duchenne Muscular Dystrophy, or DMD) are not able to express the dystrophin gene, including images of DAPI (i.e., 4′,6-diamidino-2-phenylindole, a fluorescent stain that binds strongly to A-T rich regions in DNA), dystrophin and overlay thereof, taken at 50 microns (μm). The corresponding set of images of the lower row of FIG. 7 show that after injection into the patient, matched allogeneic donor stem cells induce the dystrophin expression in the recipient patient's muscle cells.

FIG. 8 is a photograph of injection of 1 million matched allogeneic regenerative cells into four muscle groups together with 0.5 million regenerative cells given intravenously (IV) in a laboratory test mouse.

FIG. 9 is a chart showing muscle strength versus treatment regimen for a single injection of stem cells into the muscles and cells given intravenously (IV). The chart shows a significant increase in muscular strength (measured as amount of time spent in a wire hanging test) in MDX laboratory mice (i.e., a strain of mice that has a hereditary disease of the muscles caused by a mutation on the X-chromosome used as a disease model for human muscular dystrophy) that have had such an injection, for (i) MDX untreated, (ii) MDX+ fresh ADSC injection, and (iii) MDX+ cultured ADSC injection; in each case, measured from a baseline, at 4 weeks, and at 2 week intervals thereafter through 10 weeks.

FIG. 10 is a light microscopy image showing that ASCs from matched allogeneic donors engraft and transdifferentiate to new skeletal muscles expressing dystrophin.

### DETAILED DESCRIPTION

Introduction: Certain aspects of the disclosure are directed to a method for recovering regenerative cells from umbilical cord tissue, not from the blood of an umbilical cord (except for certain combined applications), and the use of those cells. The use and application of stem cells recovered from the tissue of umbilical cord can be found in several previous patent applications and references (^{9,10,11}). Cells recovered from the tissue of that umbilical cord have been described^{12,13,14,15} as being capable to differentiate better than cells derived from, for example, bone marrow. These cells are able typically to differentiate into endoderm, ectoderm, mesoderm in humans, and in animals such as horses and dogs^{16,17,18}. Methods of recovery of these cells have been described¹⁹, as well as cryopreservation of these cells²⁰. Importantly, however, certain aspects of the present invention provide an effective means of recovering a high number (population) of such cells, such as greater than typically 1 million cells per gram of umbilical cord tissue, not heretofore accomplished by the prior methods and techniques. In such aspects, about 10% or more of the cells recovered are able to form colonies, which indicates their stemness (i.e., an essential characteristic of a stem cell that distinguishes it from ordinary cells) and identify them as being primarily pluripotent stem cells.

One aspect of the present invention is to use the umbilical cord tissue-derived stem cells from a donor in a matched allogeneic transfer into a recipient in order to alleviate issues relating to the age-related limitations of the recipient's own (old and often only slowly proliferating) autologous cells for therapeutic purposes, such as repair, regeneration, or rejuvenation of damaged, diseased, or aged tissues, or in some other instances in order to correct a genetic deficiency present in the cells of the recipient, but not present in the cells of the donor, including for example schizophrenia. The characteristics of stem cells of an aged individual include: slow replication and increased doubling time, limited differentiation capacity, shortened telomeres and a high degree of senescence that potentially—especially with advanced age and concomitant diseases such as diabetes—limits the use of these aged autologous cells.

To overcome these obstacles, aspects of the disclosure pertain to the use of cells derived from umbilical cord tissue of one individual (donor) in another individual (recipient). Characteristics of these umbilical cord tissue-derived cells include, in comparison to aged cells: high genetic stability, a low degree of DNA breaks, a low degree of methylation, a fast doubling time, and a high and fast and efficient rate of differentiation into all three lineages of endoderm, ectoderm, and mesoderm. In addition, a specific genetic mutation present eventually in the cells of the recipient is not present in the cells of the donor and, therefore, these umbilical cord tissue-derived cells are, when used in allogeneic transfer, able to alleviate conditions associated with the genetic mutations in the recipient. Typically, such cells are used under the conditions that the immune surface characteristics of HLA (in humans, also called MHC, or major histocompatibility complex, constituting a set of cell surface molecules encoded by a large gene family that controls a major part of the immune system in all vertebrates), MHC1 (i.e., one of two primary classes of MEW molecules and found on nearly every nucleated cell of the body, their function being to display fragments of proteins from within the cell to T cells; healthy cells will be ignored, while cells containing foreign proteins will be attacked by the immune system) and MHC2 (i.e., MHC class II molecules constituting a family of molecules normally found only on antigen-presenting cells such as dendritic cells, mononuclear phagocytes, some endothelial cells, thymic epithelial cells, and B cells) characteristics are matched between umbilical cord donor and recipient, in procedures where the donor is the umbilical cord donor and the recipient is other than the donor and in need of administration of stem cells.

The cells can be administered in the recipient either intrathecally, intravenously, intra-arterially, into ducts such as the ductus pancreaticus, into the spinal fluid, or directly injected into tissue such as subcutaneous structures or organs such as heart, liver, kidney, brain, joints, bone or lungs. Also, an application to the epithelial layers or mucosa of, for example, the nose, mouth, or lungs, is considered.

In certain prevalent aspects of the invention, allogeneic therapies are employed. In the case of intended or desired allogeneic use of cells from an umbilical cord donor, the immune surface characteristics of these cells can be predicted by analyzing the HLA markers/type of the umbilical cord donor. Typically, this is accomplished by drawing a tiny sample of blood, of nucleated cells, or other suitable bodily fluid from the donor, such as in an amount of 100 microliters, and in the example of blood, from the umbilical cord, a vein, an artery, or an ear lobe of the donor, to determine the typical surface antigen structure HLA, HLB (i.e., human B cell differentiation antigen), DR (HLA-DR is an MHC class II antigen that maps to chromosome 6) and that even more finely serve to characterize the immune surface cell type of the donor. This information is usable to predict the future immunotype of the umbilical cord tissue stem cells when they are injected into a recipient. Typically, stem cells do not express the HLA, MHC1, or MHC2 markers, but when they differentiate in the new microenvironment into adult tissue cells of the recipient, they commence to express these markers. The matching of the markers of the recipient with the markers of the donor is important to avoid rejection of the differentiated cells or graft vs. host disease.

In instances where autologous therapy is to be performed, the umbilical cord donor is to be the recipient of the cells derived from the umbilical cord. In such a situation, the donor is known or found at birth to have certain abnormal conditions such as may have resulted from the pregnancy, birth or delivery-related complications. These may include but are not limited to cerebral hypoxia, cerebral palsy, or low APGAR (Activity, Pulse, Grimace, Appearance and Respiration) score attributable to ischemic complications during a delivery. In any such event, issues of immune matching or need therefor are moot because donor and recipient are one and the same. The same holds true if the umbilical cord tissue-derived cells are initially frozen and later thawed to be administered to the donor of the cord at some time, possibly years after birth, to treat conditions such as autism, allergies, muscle, joint or soft tissues diseases.

Typically, umbilical cord is a discarded material and, in principle, is available from every newborn delivery. The collection of umbilical cords in larger numbers allows for the establishment of a bank that incorporates cells that have been recovered from a multitude of umbilical cords and a database identifying respective immune-matching data obtained from each donor's mature cells, preferably, white blood cells or other nucleated cells, that could be used to predict the future differentiation of those umbilical cord tissue-derived cells when administered in therapeutic indications. Typically, with a standard matching of HLA markers, about 10-50 thousand donors, depending on the ethnic diversity of a population, are required to find a 100% HLA match between donor and recipient. In contrast, adaptive matching through the use of umbilical cord tissue-derived cells and umbilical cord blood-derived cells obtained from the same donor, and cryostored in a bank, can reduce this number to 3000 to 5000 donors to obtain a 100% immune-compatibility HLA matching between donor and recipient.

For storage of umbilical cord tissue-derived cells, freezing and cryopreservation and thawing of cells have been part of an established method. Even cells that have been stored at temperatures colder than −130° C. (theoretically, for 50 years) can be recovered without damage to the cells themselves, except where damage is incurred during the freezing process or the thawing process, which is readily preventable by following known proper procedures. Therefore, banked umbilical cord tissue and tissue-derived cells, as well as umbilical cord blood and blood-derived cells can be preserved for therapeutic and diagnostic purposes. HLA typing of recipients for identification and retrieval of stored cells with matching markers from the bank(s) becomes an efficient, safe and effective technique highly likely to produce positive results. For example, hematopoietic progenitor cells with CD34 marker obtained in this manner engraft successfully with bone marrow in cancer patients.

In certain aspects of the invention, both the umbilical cord tissue and the cells therefrom are separated into multiple vials, to allow thawing of the different vials of the like cells at different times. Since these cells have a very fast doubling time of 24 hours or less, it is even sufficient to freeze vials of one million cells or small samples of the umbilical cord tissue individually that, if subjected to a cell culturing in a dish or bioreactor, would allow recovery of more than 100 million cells within less than 10 days. Due to the high genetic stability of these cells, 7 or 8 doublings will typically not induce any kind of genetic aberration or genetic instability. The freezing and application of these cells at a later point in time has a further potential to regenerate not only the cells of the donor in the form of an autologous cell transfer, which has a complete immunological match, but also an enhanced probability to be available to immunologically matched family members for regenerative purposes or use in the event of genetic or acquired diseases. The chance that these cells are an immunological match is 1 out of 4 if used for siblings and very high that they could be used for other family members such as parents, grandparents and cousins.

Diseases and disorder amenable to therapy by administration of umbilical cord tissue-derived stem cells described herein include: Alzheimer's, Parkinson's, neurodegenerative diseases such as Multiple Sclerosis (MS), Duchenne Muscular Dystrophy (DMD), Multi System Atrophy (MSA), Amyotrophic Lateral Sclerosis (ALS), osteoporosis, heart failure, limb ischemia, cerebral hypoxia or perfusion defects, stroke, muscle wasting, renal insufficiency, liver diseases and insufficiency, the whole spectrum of genetic disorders including rare storage diseases, osteogenesis imperfecta, aplastic anemia, myelodystrophy, Hemophilia, Down Syndrome, Schizophrenia, Hematopoietic diseases with an underlying mutation such as for example JAK2 (Janus kinase 2 is a non-receptor tyrosine kinase implicating in signaling by members of the type II cytokine receptor family), orthopedic conditions such as diseases of joints, cartilage defects, spine disease, bone fractures, tendon diseases, skin burns, diabetes, cerebral palsy, lung fibrosis, asthma and chronic obstructive lung disease, atrophy of the nose mucosa, vascular associated diseases such as arterial peripheral vascular disease, Crohn's Disease, Colitis ulcerosa, Lupus and other autoimmune diseases, HIV including modified cells to provide a mutant CCR5 receptor (i.e., C—C chemokine receptor type 5, also known as CD195, is a protein on the surface of white blood cells that is involved in the immune system as it acts as a receptor for chemokines) or combinations thereof. Also, these cells are ideal for cancer therapy in a matched or unmatched allogeneic transfer, especially after they have been genetically modified to serve as a “Trojan Horse” and when given to a patient unload their cancer therapeutic freight within the tumor.

Delivery of matched allogeneic cells may also be used to treat a growing fetus that has a genetic disease. As molecular diagnostics have significantly increased over the last years genetic diseases of the fetus are visible not only by amniocentesis, but also from DNA from the blood in the mother and from circulating erythrocytes—which are nucleated of the baby—in the blood of the mother, to determine specific genetic diseases in the growing fetus. At present, no therapy is available for these conditions. Matched allogeneic umbilical cord tissue cells from a donor can be injected during the pregnancy into the umbilical cord arteries of the growing fetus when it is located in the uterus of the mother. This injection contains matched allogeneic cells from a donor in which genetic determination has shown that the specific mutation of the growing fetus, is not present. Repetitive injections of those cells will help, that the transplanted stem cells will partly take over the function of the modified gene and restore diseased gene related protein expression in the baby. It is a known practice to locate the umbilical cord inside the uterus by ultrasound and with a transcutaneous injection into the circulation of the growing fetus to apply those stem cells from a donor. If this were performed in a matched allogeneic transplant way, graft versus host disease must be considered.

Another aspect of the disclosure is to further select, recover and distribute modified umbilical cord tissue-derived cells with a specific genetic mutation with or without culturing or freezing and thawing. A sample of more than 100 million cells of umbilical cord tissue-derived regenerative cells can serve a large population of diseased or aged persons. For example, cells that have a mutation in CCR5 can be used to treat patients with HIV. Cells that, for example, lack the same genetic mutation as it is present in the future recipient, are valuable to be used to correct inborn genetic diseases. In this case, the cells are used in a matched allogeneic manner. These cases are, for example, the transfer of umbilical cord tissue-derived cells from one donor sibling without a genetic disease to a recipient immune-matched sibling with an existing genetic disease such as DMD or Hemophilia with, for example, Factor 8 deficiency. Injection of umbilical cord tissue-derived cells from a donor having no genetic disease into an allogeneic-matched recipient having a genetic disease such as Hemophilia results in the production of Factor 8 by newly formed endothelial cells derived from the transplanted umbilical cord tissue-derived cells in the recipient with the genetic defect.

As another example, in certain instances therapy for genetic diseases is achieved through a method based on the absence of a genetic mutation in the donor umbilical cord tissue-derived cells to treat a matched recipient with a genetic disease. For example, one method herein disclosed is the identification of a genetic deficiency such as a mutation in the dystrophin gene that causes DMD or Becker muscular dystrophy; isolation, storage and characterization of umbilical cord tissue-derived cells from a donor that lack the identified genetic mutation; matching of the HLA, MHC1 and/or MHC2 surface immune-markers of a potential recipient with the umbilical cord tissue-derived cells of the donor; and application to the recipient of those umbilical cord tissue-derived cells that are genetically normal compared to the cells of the recipient for therapeutic and diagnostic purposes. In case of DMD, for example, injected donor umbilical cord tissue-derived cells need to have at least one normal X chromosome that does not carry the genetic mutation.

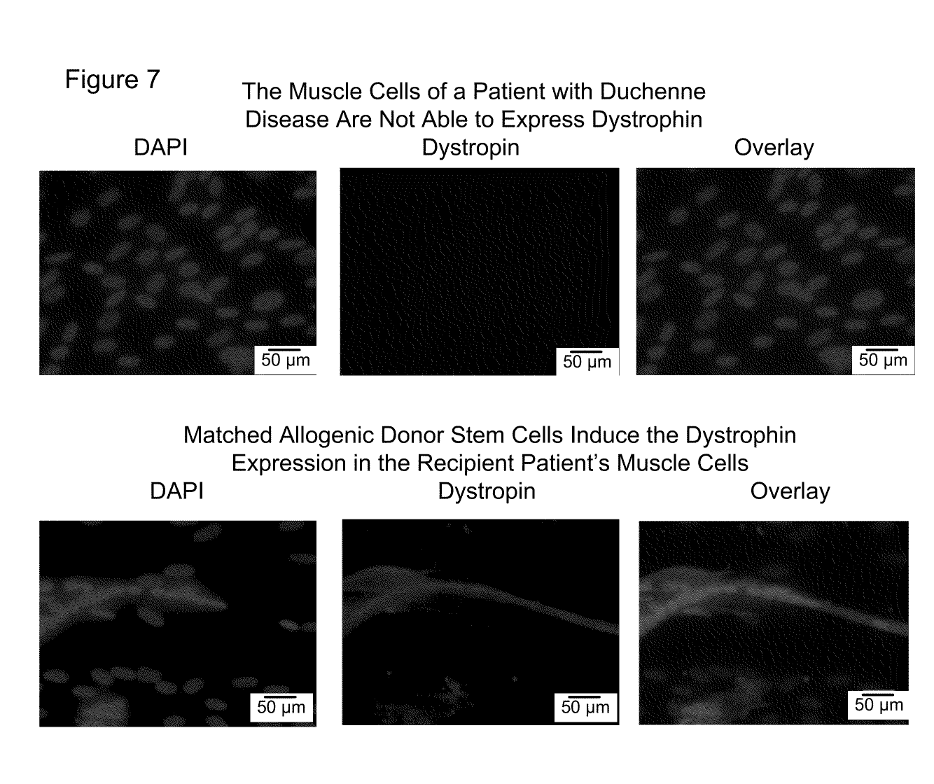

Injection of matched allogeneic cells from a donor in a recipient with DMD is highlighted in FIG. 7. The muscle cells of a recipient with DMD disease are typically not able to express dystrophin. As can be seen, only the nuclei of the muscle are stained, and staining for dystrophin shows no presence of the respective dystrophin protein, which is typical of this disease (FIG. 7, upper row). Matched allogeneic stem cells from a donor without the genetic disease are able to induce the dystrophin expression in the recipient's muscle cells, as shown in the overlay on the right side of FIG. 7, lower row. Here, multi-nucleated cells form between the recipient's nuclei and the nuclei from the matched allogeneic cells that do not carry the mutation, thereby inducing a protein expression of the missing dystrophin.

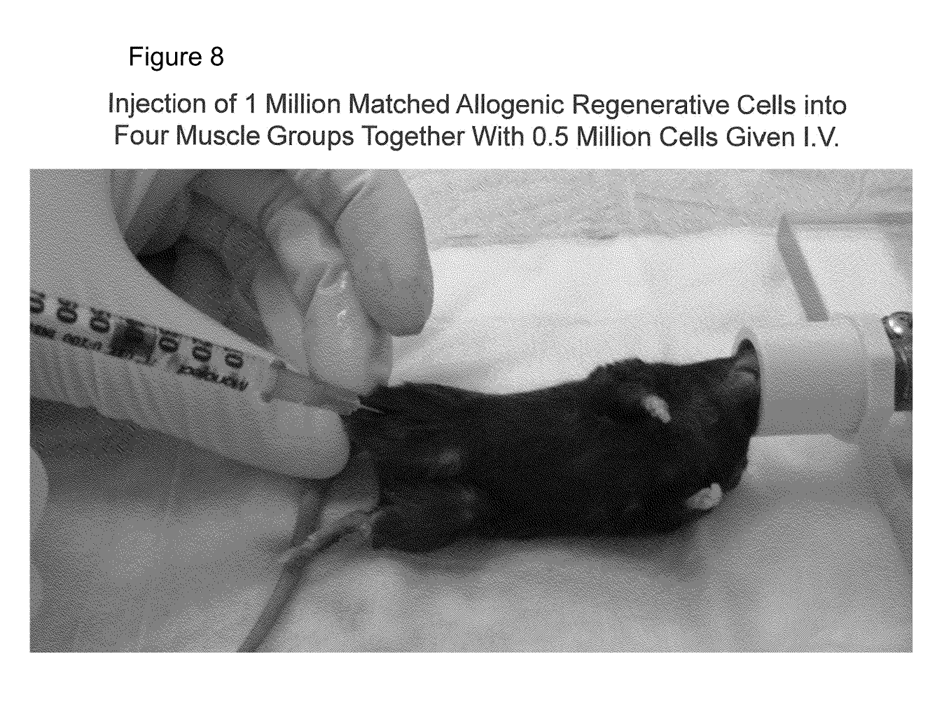

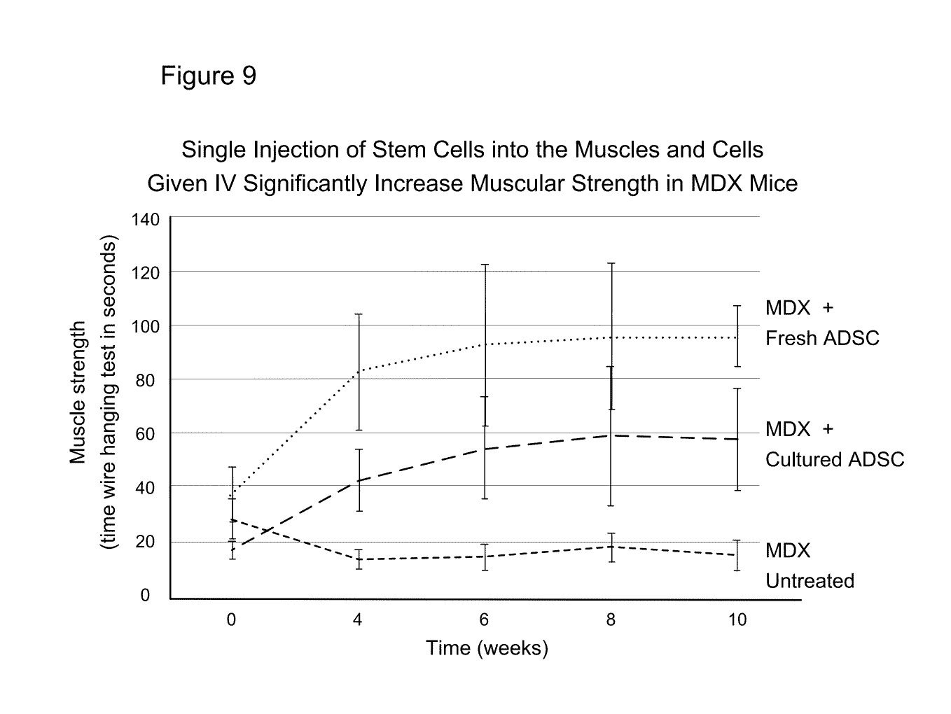

After treatment of the recipient's diseased muscle cells that have the DMD genetic mutation with donor umbilical cord tissue-derived cells that have a normal genetic profile and do not have the DMD mutation, the recipient's muscle cells are able to show an expression of the missing dystrophin protein and are able to convert this into muscular strength, as is evidenced by the following experiment. In a MDX mouse that resembles the human genetic deficiency of dystrophin, one million regenerative stem cells have been injected into the four major muscle groups of fore and hind limbs (FIG. 8). In addition, half a million of these normal cells without the genetic muation are given intravenously to the respective mouse. Before the injection, the mice have been subjected to a wire hanging test in order to evaluate their musclar strength. Normal mice (wild type) are able to hold for 300 seconds when hanging on a thin wire until they fall down. The mice with the genetic disease (MDX untreated) are able to hold up for 20 seconds at baseline and over time the musclar strength is reduced to about 14 seconds after 10 weeks (FIG. 9).

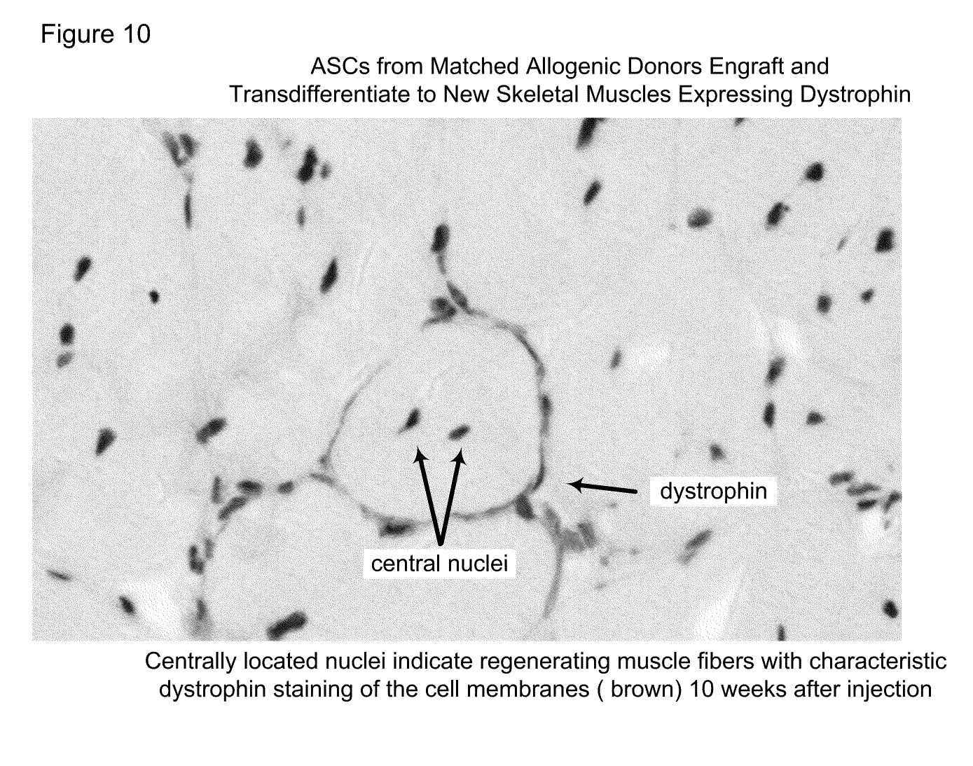

Injection of fresh, cultured, or frozen and thawed matched allogeneic cells into the muscles and given intravenously (IV) effects a significant increase in muscular strength enabling those genetically compromised mice to hold themselves on the wire about 100 seconds until they release and fall. The gradual increase of muscular strength over time indicates that the cell transfer is therapeutically beneficial and converted into building new muscles respectively inducing a dystrophin expression in already existing muscles (as FIG. 10 shows). Central nuclei in the histology section indicate the formation of new muscles. The transplanted allogeneic matched donor cells are able to differentiate and able to express dystrophin, which can be shown 10 weeks after the injections.

The application of allogeneic umbilical cord tissue-derived cells in patients in need of the cells is typically done repeatedly. In this way, deficient cells of a recipient are replaced more and more and over time and with normal stem and progenitor cells of a donor. In order to prevent a rejection or GVH (graft versus host) phenomenon, the cells are applied in a matched allogeneic transplant mode. Therefore, common standard and known methods such as determination of the markers of HLA-A, HLA-B, HLA-C (this belongs to MHC class I heavy chain receptors, the C receptor being a heterodimer consisting of a HLA-C mature gene product and β2-microglobulin), HLA-D or B1, HLA-DQB1 (this is a major histocompatibility complex, class II, in which DQ beta 1 is a human gene and also denotes the genetic locus that contains this gene^{I}; the protein encoded by this gene is one of two proteins that are required to form the DQ heterodimer, a cell surface receptor essential to the function of the immune system) and further molecular biological typing is performed in order to find the right high probability of an allogeneic matched donation, and the typing including the determination of antibodies is performed. Aside from the standard typing, there is also an enhanced epitope typing applicable to reduce the need for higher numbers of donors to find a perfect or acceptable match of HLA immune markers for banking cells of typically about 30 thousand individuals to potentially down to only three thousand samples if the ethnic group included in the matching is restricted to certain geographic areas of ancestral lineage.

Use of both cord tissue-derived cells and cord blood-derived cells from the same donor applied together to a recipient provides additional beneficial effects. While cord tissue-derived cells are truly pluripotent and are able to differentiate into any of the three germ layers with guidance from the microenvironment, cord blood-derived cells very rarely have this pluripotency. The yield of nucleated cells from a donor's specimen of cord blood is very high (500 to 750 million nucleated cells), but only every second donor's cord blood contains pluripotent cells, and if they are found at all, then only in very limited numbers; so typically, culturing is required before usage if it is the aim to transfer pluripotent stem cells derived from cord blood. However, cord blood cells contain hematopoetic progenitor cells that are capable of differentiation into bone marrow cells. This makes them available in case of an ablation of the bone marrow for cure of hematopoetic malignancies, such as a bone marrow transplantation.

The two cell types are complementary, since it has been shown that the reconstitution of an ablated bone marrow is easier, when not only hematopoetic progenitors are transplanted, but when they are transplanted together with pluripotent stem cells. In accordance with this invention, this is achieved by administering umbilical cord tissue-derived cells in combination with umbilical cord blood-derived cells from the same donor.

The particulars disclosed herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings making apparent to those skilled in the art how the several forms of the invention may be embodied in practice. This includes methods and apparatus of the applications and other publications incorporated by reference in their entireties herein.

Umbilical cord represents and contains an abundant source of perinatal, plastic adherent mesenchymal stem cells (UC-MSCs). UC-MSCs typically exhibit robust stemness and strong immunosuppressive and regenerative effects in vivo. Processing of umbilical cord tissue by the method of the invention efficiently isolates large numbers of fresh nucleated umbilical cord regenerative cells (UC-RCs) that exhibit similar characteristics of UC-MSCs. This can alleviate the need for culture expansion to obtain large numbers of cells required for clinical application especially if the cells are used in the peri-natal setting immediately after delivery in order to be used for therapy of pregnancy, birth or delivery-related complications, such as those resulting from cerebral hypoxia, cerebral palsy or low APGAR conditions.

Dissociation is achieved with a blend of collagenase and neutral protease with agitation at 37° C. to 40° C. in a semi-automatic system. The average yield is about or more than one million nucleated cells/gram tissue with more than 80% viability. Viability greater than 85 to 95% has been achieved. The procedure to recover cells from umbilical cord tissue utilizing the method of the invention is often less than 30 minutes for umbilical cord segmentation and less than 180 minutes for processing and recovering a cell preparation. Indeed, the time for recovering a cell preparation can be less than 120 minutes, and even less than 60 minutes if the tissue is finely minced and prepared before processing thereof. Quickly obtaining a large number of regenerative cells that have pluripotent differentiation capacity without the complexity and risks of culture expansion simplifies and expands the use of regenerative cells in clinical therapeutic as well as research applications.

This is especially true for so-called immediate “point of care” processing and applications, i.e., the recovery of regenerative cells immediately after delivery, processing and reapplication to the baby in need. Diseases or disorders of cerebral hypoxia, low APGAR values and cerebral palsy following complicated deliveries are exemplary of those calling for processing the umbilical cord tissue in close proximity to the delivery site. This allows for administration of umbilical cord tissue-derived cells to the umbilical cord donor within 24 hours of the donor's delivery. The umbilical cord tissue-derived cells can be aliquoted, to allow a first aliquot to be administered within 24 hours of the delivery of the donor, and additional aliquots to be administered subsequently. The subsequent cell applications are part of a therapeutic regimen for the disorders mentioned immediately above. Also, diagnosed inborn genetic diseases might benefit from an early stem cells transfer, in an autologous or matched allogeneic way. The cells can be administered intravenously, intra-arterially, intrathecally, into the mucosa or surface of the airway system, including, for example, mouth, nose or lungs, or into a duct, spinal fluid, tissue or organ.

Mesenchymal stem cells (MSCs) typically define a population of cells with the potential to differentiate into all three germ layers in vitro. In vivo, MSCs promote tissue regeneration and healing, and elicit potent immune-modulatory effects. These characteristics of MSCs have generated significant interest in their use for cell-based regenerative therapies. MSCs reside in all tissues in the body and can be obtained from primary cell isolates of different sources in adult tissues and fetal tissues since they adhere to plastic in cell culture. While isolation methods for adult tissues have been mainly established for adipose tissue and bone marrow, the main source for fetal tissue is the umbilical cord (UC). Due to their origin from fetal tissue, the perinatal MSCs in the umbilical cord exhibit higher stemness and immune-modulatory properties compared to MSCs originating from adult tissues without the limitations and negative aspects of embryonic stem cells.

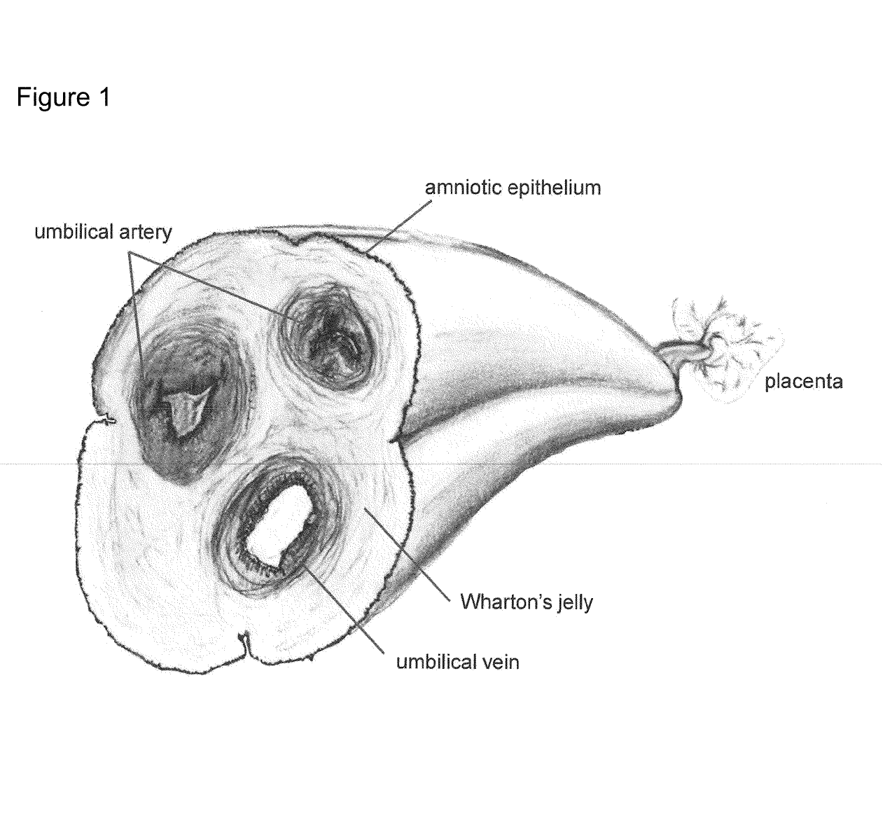

The umbilical cord of most mammals consists of two arteries and one or two veins (matrix) that are embedded in a jelly-like ground substance of hyaluronic acid and chondroitin sulfate (FIG. 1), named Wharton's jelly after Thomas Wharton, who first described it in 1656. Recent research has shown that the Wharton's jelly contains plastic adherent MSCs (UCT-MSCs) that exhibit properties close to but different from embryonic stem cells. In addition to their potential for differentiation to cells of all three germ layers. UCT-MSCs have a higher rate of proliferation and may have more prolonged potential for self-renewal compared to adult MSCs. It has been shown that the presence of longer telomeres in UCT-MSCs is responsible for this increased capacity for self-renewal prior to senescence. Due to their close developmental relationship to embryonic tissue (without the teratoma risk of embryonic cells), UCT-MSCs possess broad plasticity and immune-modulatory characteristics in vivo. However, in contrast to embryonic stem cells, UCT-MSCs have important and critical advantages, including absence of ethical concerns, abundant availability since the umbilical cord is part of every newborn delivery and is typically discarded material, and produce neither tumor formation nor carcinogenicity. Moreover, the low immunogenicity, ability to home in on sites with ongoing tissue inflammation, and ability to promote neovascularization and tissue regeneration of UCT-MSCs provide, in appropriate instances, a compelling rationale for allogeneic transplantation. Since Wharton's jelly and the matrix provide a rich perinatal source of UCT-MSCs, a method for efficiently isolating large numbers of cells quickly serves to advance the use of UCT-MSCs in research and clinical applications.

Isolation of MSC from the Wharton's jelly was first described by McElreavey et al. His group cultured minced Wharton's jelly without prior enzymatic processing up to two weeks in order for the UCT-MSCs to migrate out of the tissue and adhere to the culture dish. This method yielded a relatively low initial number of cells and required more extensive culture expansion after the initial two-week period. Currently, MSCs are isolated from the umbilical cord by the use of collagenase followed by a selection for plastic adherent subpopulation in cell culture. As shown in Table 1 all published protocols for UCT-MSCs isolation commonly result in a relatively low cell yield and require some kind of cell culture expansion to obtain the high cell numbers that are recommended and required for preclinical studies and, especially, clinical usage.

TABLE 1

| | | | | | | | Cells/ | |
|---|---|---|---|---|---|---|---|---|
| Date | Author | Journal | Material | Duration | | No. | cord | Viability |
| January | H. Wang et al. | Stem Cells | WJ | ≈18 | hrs | 30 | — | — |
| 2004 | | | | | | | | |
| November | M. Bailey et al. | Tissue | WJ | ≈1 | week | 4 | — | — |
| 2007 | | Engineering | | | | | | |
| March | D. Campard et | Gastroenterology | WJ | ≈1 | day | 15 | 1.22M ± | — |
| 2008 | al. | | | | | | 1.09M | |
| | | | | | | | cells | |
| December | N. Tsagias et al. | Transfusion | cUC | ≈4.5 | hrs | 12 | 0.96M | 81% |
| 2010 | | Medicine | | | | | cells | |
| March | I. Christodoulouet | Stem Cells | WJ | ≈2.5 | hrs | 5 | 2.28M ± | 94.3% ± |
| 2013 | et al. | International | | | | | 1.55M | 2.2% |
| | | | | | | | cells | |
| October | J. Hua et al. | Cell Biology | WJ | ≈1-4 | hrs | — | Explant: | — |
| 2013 | | International | | | | | 0.042-0.0201M | |
| | | | | | | | cells | |
| | | | | | | | Enzyme: | |
| | | | | | | | 0.003-0.027M | |
| | | | | | | | cells | |

Given that these protocols entail several days, weeks to months of culture expansion, differences in cell characteristics including regenerative potential, induction of chromosomal changes and changes in cell surface antigens, the cells are rendered somewhat less effective and useful when used in clinical setting. In addition, expansion in culture has potential for exposure to xenogenic proteins and contamination as well as additional complexity and cost associated with the previous described methods. Also, immediate cell therapy in the peri-delivery timeframe of 12 to 48 hours is not feasible.

Ex vivo expansion and differentiation of stem cell populations is considered to be substantial manipulation by the United States Food and Drug Administration and the European Medicines Agency. For adipose tissue, fresh preparation of nucleated regenerative cells using a novel semi-automated system (generally referred to as the stromal vascular fraction or SVF), enables high cell yield and therefore obviates the need for cell culture expansion to obtain a high number of pluripotent cells to be used immediately after delivery or later on after freezing, storage and thawing in an autologous or matched allogeneic matter. The protocol of the present invention aims to facilitate the ability to efficiently and more rapidly isolate fresh regenerative cells from umbilical cord (UC-RCs or umbilical cord regenerative cells) with high cell yield to enable potential therapeutic application without the need for culture expansion.

This protocol of the invention has been developed based on a protocol for isolating the SVF from adipose tissue without ex vivo cell culture expansion. The SVF resulting from the aforementioned isolation process has been designated as a non-ATMP (i.e., not an advanced therapy medicinal product) by the European Medicines Agency when used for regeneration, repair, or replacement of weakened or injured subcutaneous tissue. (EMA/129056/2013 is the scientific recommendation on classification of advanced therapy medicinal products—Summary for Public Release for “Adult Autologous Regenerative Cells for Subcutaneous Administration”). This designation includes a determination that the SVF preparation was not subjected to a substantial manipulation. Additionally, this protocol has been successfully used for the isolation of SVF from debrided skin and adipose tissue from burn victims as well as from equine lipoaspirate samples.

### Non-Limiting Examples:

In an exemplary method, umbilical cord tissue is processed to recover regenerative cells therefrom. Tissue dissociation is achieved using a mammalian origin free, optimized blend of collagenase and neutral protease, and mechanical processing at elevated temperature in a novel semi-automatic system. This combination results in a high viability of the recovered cells and high yields of UC-RCs in shorter time with less operator involvement. The protease can be produced via a recombinant process.

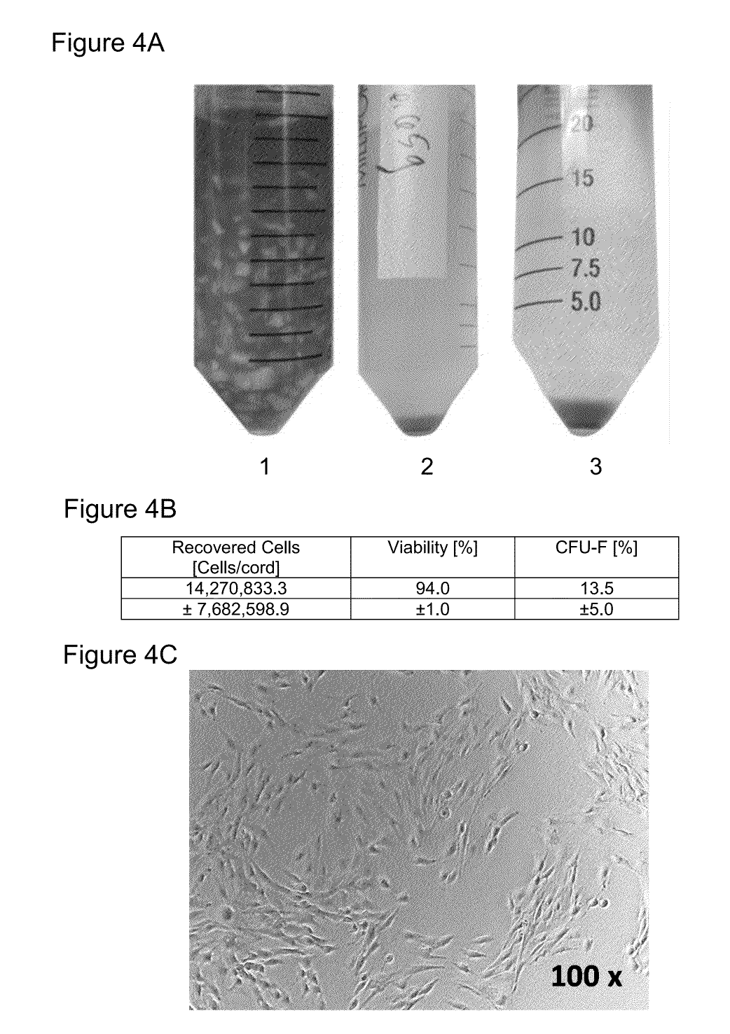

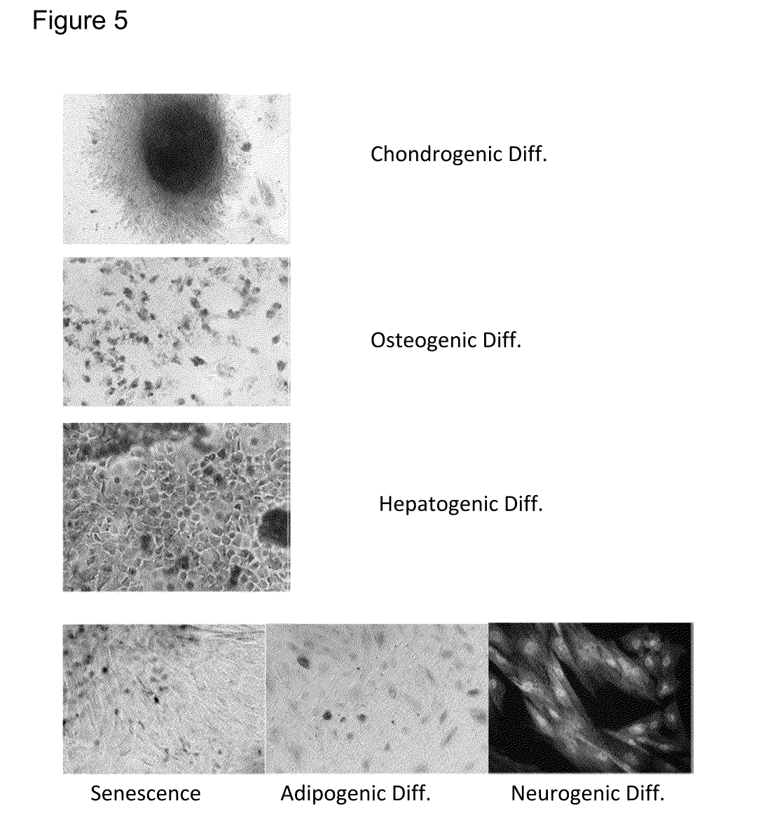

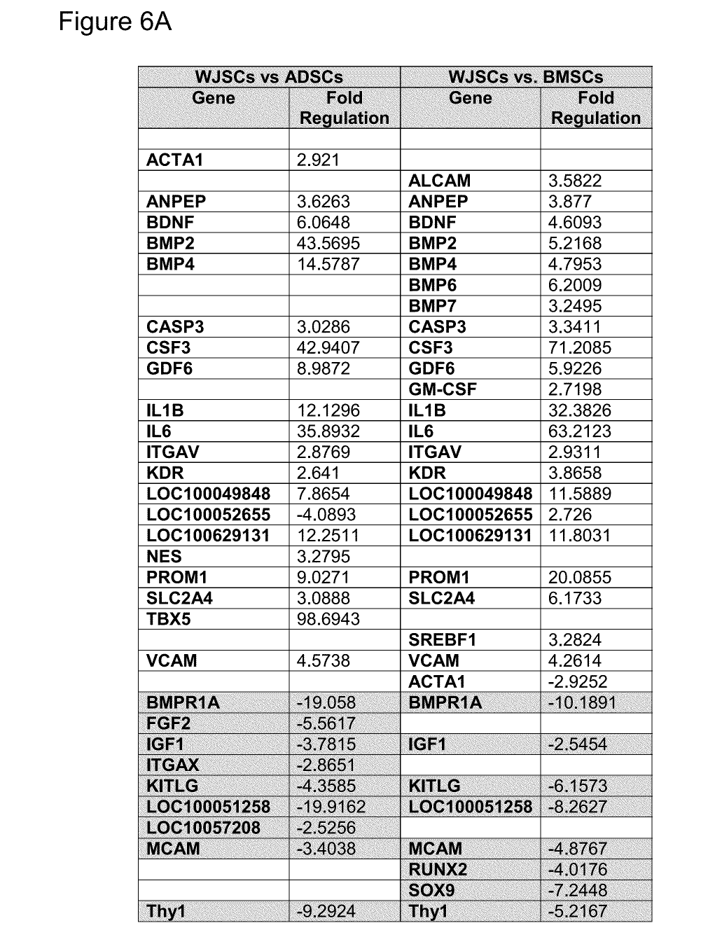

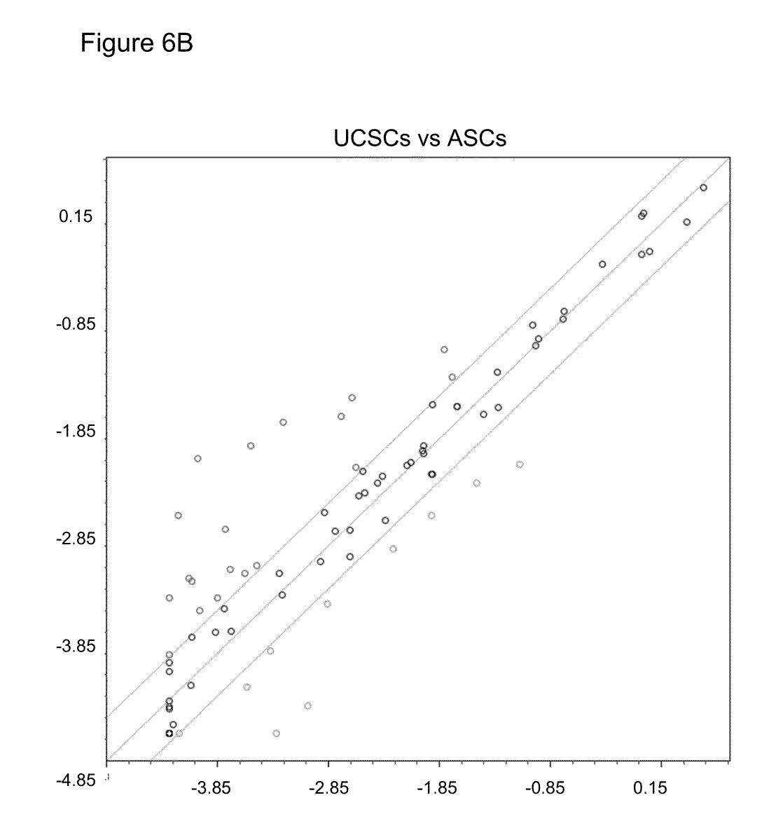

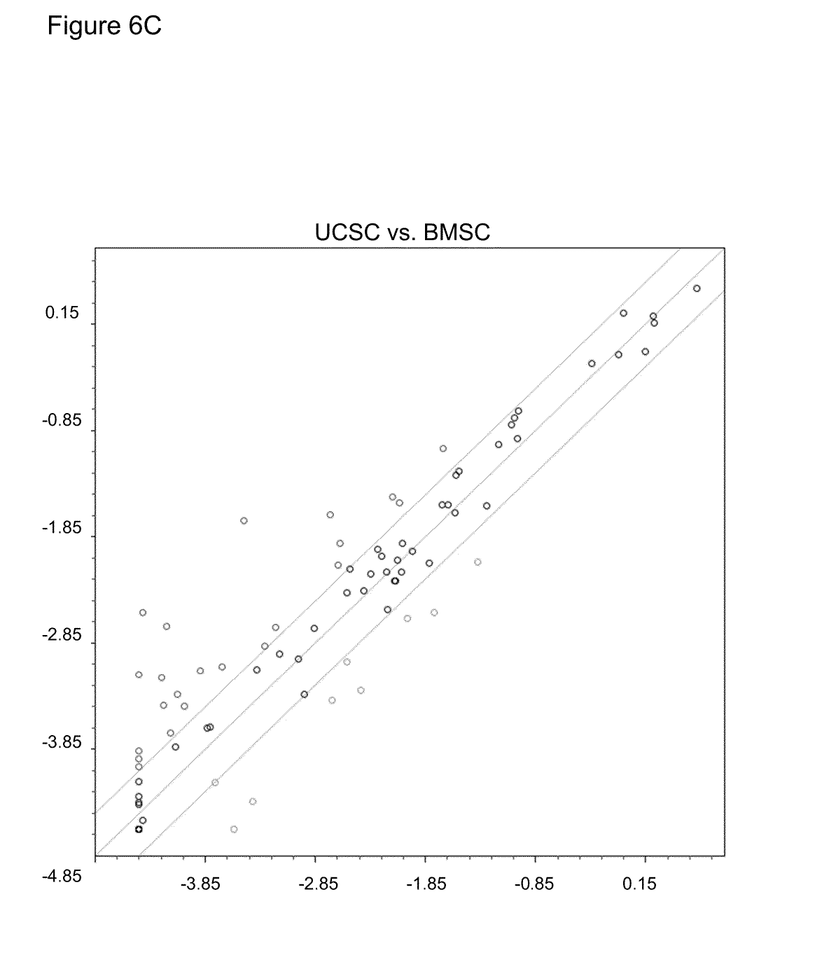

In this exemplary method, umbilical cords are harvested; then washed and disinfected; and segmented to obtain umbilical cord tissue segments including matrix, stroma, and Wharton's jelly. The umbilical cord tissue segments may be minced for subsequent processing as described presently to obtain stem cells therefrom. In order to compare the regenerative cells acquired from umbilical cord tissue by methods of the present invention to previously published results of others, the plastic adherent fraction (UCT-MSCs) of the regenerative cells from umbilical cord tissue was characterized. Initial cell yields were quantified and cultures of the primary UCT-MCSs from passage 0 to passage 2 (the latter is the third culture following the isolation of cells from tissue; also called the tertiary tissue) were characterized by assessing their colony forming potential (FIG. 4). Additionally, their capacity to differentiate along all three germ layers, including cell types of the mesoderm such as osteocytes, chondrocytes, adipocytes, cell types of the endoderm such as hepatocytes, and cell types of the ectoderm such as neurons was assessed (FIG. 5). Gene expression profiling by RT-PCR array for MSC-specific genes was also performed (FIG. 6). RT-PCR, or reverse transcription polymerase chain reaction, is one of the many variants of PCR, and this technique is commonly used in molecular biology to detect RNA expression levels. RT-PCR is used to quantitatively detect gene expression through creation of complementary DNA (cDNA) transcripts from RNA.

Umbilical cords were collected following parturition from normal full term pregnancies with unassisted or assisted delivery. In this aspect of method, umbilical tape was tied around the cord in 2 places; adjacent to where the cord breaks or is severed from the baby and of maximum useable length toward the placenta. The ligations were placed to limit contamination into the lumen of the cord. The isolated portion of the cord between the ligations was placed on a clean surface and any visible gross contamination physically removed with sterile surgical instruments and gauze sponges. In this aspect of this disclosure, the cord was rinsed by shaking in a 1.5 L bottle with sterile 0.9% saline solution three times and then washed with fresh solution containing disinfectant, antibiotic, and antifungal substances, and then placed in cold (4° C.) saline solution until processed. The cord then can be soaked in a solution containing substances to begin dissociation of the umbilical cord before the cord is processed within 24 hours after collection.

After collection, umbilical cords, in this aspect of the method, are prepared by washing the tissue in a wash solution of Penicillin and Streptomycin (10 IU and 10 μg/mL), gentamycin (2.5 μg/mL) and amphotericin B (250 ng/mL) to Phosphate Buffered Saline (PBS). The samples in this aspect of this disclosure were washed twice with 1-3% hydrogen peroxide in sterile water and three times with the prepared wash solution and finally rinsed with saline solution.

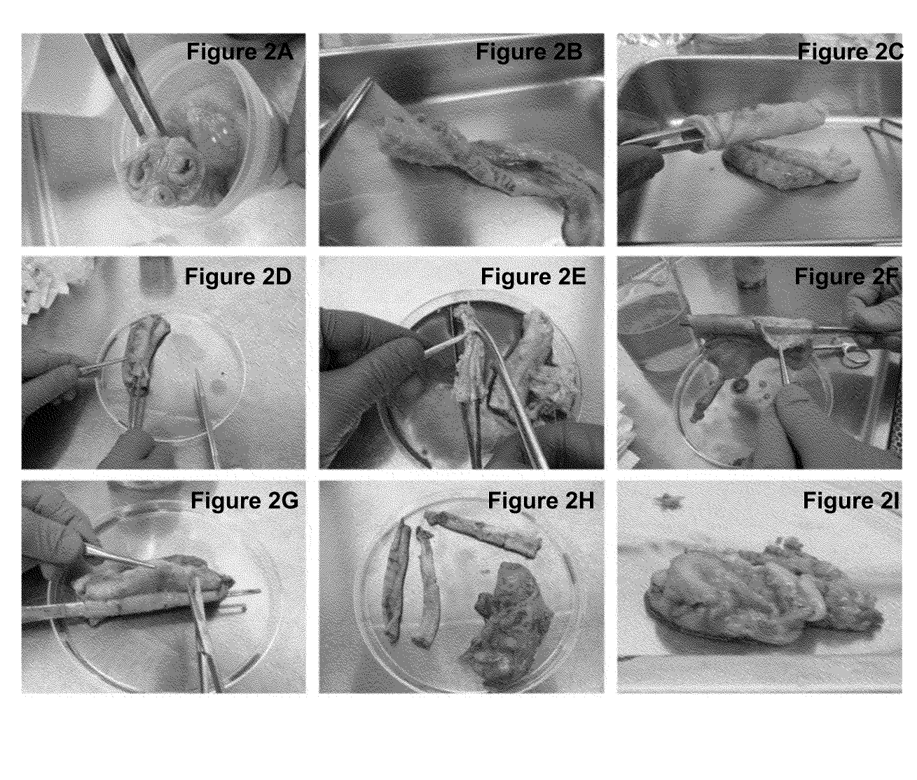

The umbilical cord contains two arteries (firm, thick walled) and one vein (pliable, thin walled) surrounded by the Wharton's jelly, which insulates and protects the umbilical cord vessels (see FIG. 1, FIG. 2A). The umbilical cord in this aspect of the method is segmented. The tissue samples are placed on large sterile petri dishes for dissection, and the thin squamous epithelium is removed and discarded (see FIG. 2B).

The remaining tissue is composed of the vessels, stroma, and Wharton's jelly. The fascial plane around the vessels is then dissected to separate the surrounding Wharton's jelly from the vessels. The vessels can be then discarded to isolate only the Wharton's jelly. This is preferred in cases where a low processing time is required or desired since processing of the vessel to release the cells typically takes longer than the processing of the Wharton's jelly for cell recovery. The Wharton's jelly is placed onto a separate dissecting plate and minced prior to further processing of the tissue. A higher degree of mincing beneficially affects the subsequent processing time. FIGS. 2A-I represent a photographic sequence of a dissection of an umbilical cord sample, illustrating in FIG. 2A, the umbilical cord anatomy; in FIG. 2B, removal of the umbilical cord amnion epithelium with a hemostat; in FIG. 2C and FIG. 2D, expansion of the umbilical cord vessels using medium size scissors to facilitate the separation of the vessel from the Wharton's jelly intervascular tissue; in FIG. 2E, FIG. 2F and FIG. 2G, stepwise dissection of the Wharton's jelly tissue from other structures such as the vessel walls is shown; in FIG. 211, umbilical cord tissue including vessel, stroma and Wharton's jelly tissue is shown after dissection; and in FIG. 2I, the resulting umbilical cord tissue is shown.

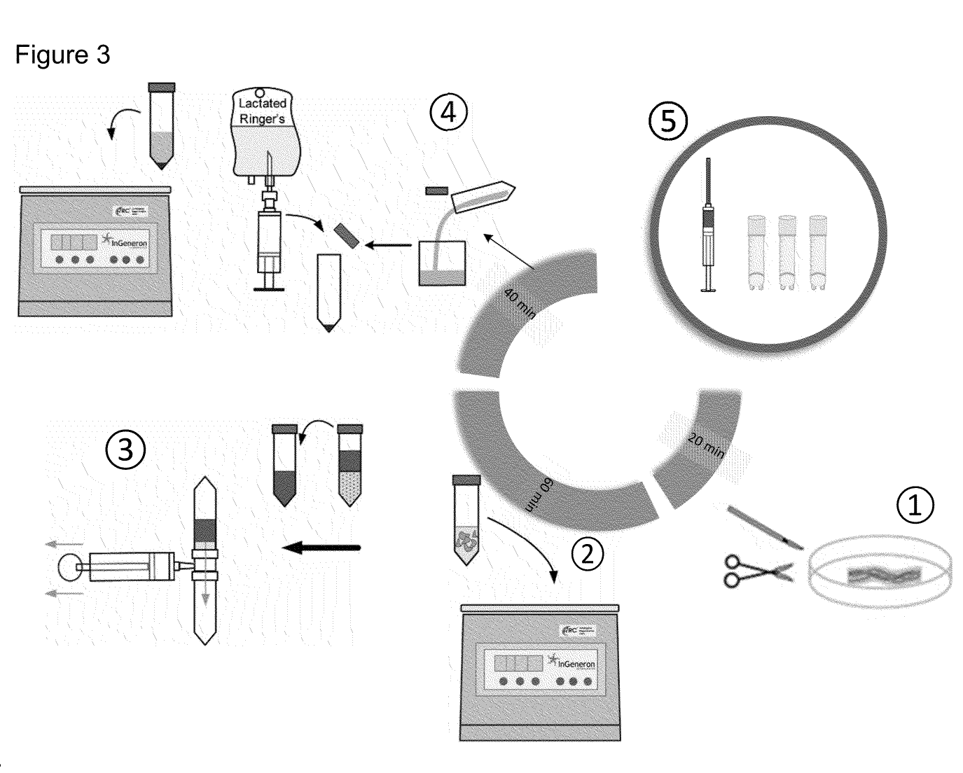

Umbilical cord regenerative cells are isolated with a pre-warmed tissue processing unit, such as an ARC™ tissue processing unit from InGeneron Incorporated, of Houston, Tex. The ARC™ tissue processing unit is disclosed in U.S. patent application Ser. No. 13/385,599 (published as US 2013/0115697) and U.S. patent application Ser. No. 13/329,142 (published as US 2012/0195863), each of which is incorporated by reference in its entirety herein. In brief, the ARC™ tissue processing unit is a specially designed unit for tissue processing and centrifugation, with the capability to lock tube holders in an inverted upright position. A lactated ringer's back was pre-heated to 37° C. (FIG. 3). Approximately 10 g-25 g each of prepared minced umbilical cord tissue respectively is placed into one or more sterile processing tubes. 25 ml of the aforementioned lactated ringers is added to each tube to reach a final volume of 35 ml-50 ml per tube.

One to five units of MATRASE™ Reagent (available from InGeneron Incorporated, of Houston, Tex.), a proprietary collagenase and neutral protease enzyme blend, is added to each gram of tissue at a concentration of 10 units/ml of solution. The sample tubes are inverted to mix the enzyme with the tissue and then placed in the processing unit described above and processed for 1-4 hours under the increased temperature environment of the inner portion of the processing unit.

Following processing, the samples are placed on a rack to allow sedimentation for 2-3 minutes in this method (FIG. 3). The supernatants are collected and transferred to sterile processing tubes. The tissue slurry is filtered through a 100 micron filter. The cells are concentrated by centrifugation of the filtrate at 600×g for 3 to 5 minutes (FIG. 4). The pellets are re-suspended thereby making a cell suspension in saline solution to assess cell viability and cell counts. The cell preparation can either be used immediately or cryopreserved and stored (banked) for future applications. In one embodiment, ultrasound guided injection of the umbilical cord derived cells are delivered in a matched allogeneic way. The volume of these injections should be in a range of a 100 microliter to 1 ml. Representative examples for in vitro characterization of cultured UCT-MSCs demonstrating potential for differentiation into cell types of all three germ layers, CFU-F assay, and gene expression profile are indicated in FIGS. 4-6.

### Cell Viability and Nucleated Count Assessment

As described above, the cell preparation is re-suspended in saline solution, additionally a green fluorescent nucleic stain was administered to the suspension, and the stained cells viewed under fluorescent microscopy. Trypan blue staining was used with light microscopy to visualize and count dead cells.

### Cell Cryopreservation

In order to preserve cells, the pelleted cells, in this aspect of the disclosure, are suspended by centrifugation at 400×g for 3 to 5 minutes. The supernatant gets removed, and the cells are re-suspended in cryopreservation media. The cells are then transferred to a cryovial, and the cryovial is placed on a 100% isopropanol containing cryopreservation chamber. The chamber is then placed in a −80° C. freezer overnight. For long-term storage, the sample can be, either frozen directly in or transferred to the gas phase of liquid nitrogen. In one aspect of this disclosure, the cell preparation is aliquoted and each aliquot is cryopreserved for later use.

The recovered preparation of regenerative cells can be used either directly after preparation or after thawing of cells that have been previously been frozen. For freezing and thawing the respective known technology is applied. The cell preparation is then prepared according to the specific indication. For IV application for example, the cells are diluted in an isotonic solution. The volume of the solution depends on the size of the patient. For a new born baby, 10-30 ml of isotonic solution will be connected to a vein, or an artery or prepared in a syringe for puncture of the cerebral fluid for intrathecal or intraductal application. For IV and intra-arterial application, about 2-20 million cells are dissolved in 10-20 ml solution. If the injection of cells occurs directly into an organ, such as a joint, a liver or into any other organ, the volume of the injection is considerably lower and ranges between ½ ml-5 ml, also dependent on the size of the patient. For adult patients with a body weight between 50-100 kilograms, typically 250 ml of saline solution and cells in a range between 20 and 100 million will be slowly infused over 30 minutes either into an intravenous application, or through a catheter into an intra-arterial location. Also if the cells are used for local treatment of an organ, the volume will be considerably lower in a range of 1-5 ml; that means the concentration of cells will be considerably higher. Also for intrathecal or intraductal application, the amount of fluids will be in a range of 1-20 ml. For intramucosal delivery a fluid volume of 1-20 ml is selected. For ultrasonic guided injection of umbilical cord derived cells in a match allogeneic way, the volume should be in a range of a 100 microliter to 1 ml.

As discussed above, cells can be either prepared within the site of the delivery where the umbilical cord initially is recovered, or at a later remote location. Usage is either in an autologous manner, meaning the cells are used only for umbilical cord donor, or in a matched allogeneic manner, meaning that the HLA markers of the umbilical cord donor will be matched with the recipient's HLA markers in order to avoid immunological reactions. In rare cases, when the cells are not expected to transdifferentiate but only have an indirect effect by release of cytokines, exosomes or application of regulatory T cells, then the cells can be used in a non-matched manner.

This holds true if a co-culturing of cells with the stem cells of a patient if intended, for example for rejuvenation purposes. In this case, the umbilical cord cells can be cultured in serum or serum free media, and cytokines and exosomes derived from those umbilical cord cells can be collected and administered to the patient directly. Or in a co-culture approach, the patient's own stem cells and progenitor cells—cultured in the presence of the cytokines and exosomes contained in the culture media in which the umbilical cord tissue were cultured—are collected and administered. It has been shown by respective studies underlying and supporting this application that the media in which a donor's umbilical cord cells have been cultured contains valuable substances usable for various diagnostic and therapeutic applications. For example, the media can be used in a direct way (injected into the donor or a recipient) or in an indirect way (namely, by culturing a recipient's own cells in the media derived from the culturing of the donor's cells).

One challenge of isolating regenerative cells from umbilical cord that can be re-administered to a (often elderly) patient as sterile cells is avoidance of contamination of samples during the process of obtaining the umbilical cord. Since newborns are usually vaginally delivered, contamination of the umbilical cord is inevitable. Tissue collection during warm weather conditions can expedite bacterial and fungal growth on tissue samples. Certain interventions can be employed to decrease the risk of contamination of cells during and downstream of the isolation process. The most important step is to minimize bacterial and fungal growth immediately after obtaining the tissue sample. If possible, a sterile field should be set up for working with the freshly obtained tissue sample. The umbilical cord should be rinsed with cold Lactated Ringer's or saline solution. Additional washes as disclosed above plus usage of a providone-iodine solution may also aid in reducing or depleting the bacterial and fungal flora on tissue samples.

To prevent pathogen growth between tissue collection and tissue processing the cord should then be kept chilled in Lactated Ringer's or saline solution containing 1% of Penicillin, 1% Streptomycin, 0.01% of Gentamycin and 0.2% of Amphotericin B. This antibiotic and antimycotic regimen corresponds with the typical combination in the regular growth media for primary MSC cultures. Identifying the bacterial and fungal flora of the tissue sample right after collection and testing for antibiotic resistance may help in adjusting the antibiotic regimen if microbial growth occurs during culture expansion. Additionally, incubating the cord in the MATRASE™ Reagent helps to release the stem cells in higher numbers from the cord in a shorter amount of time and to reduce the viability of otherwise contaminating bacteria and fungi.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. It should further be understood that the references, patents and patent applications disclosed herein are incorporated by reference in their entirety.

### REFERENCES

1. Zhang Y Y, Yue J, Che H, Sun H Y, Tse H F, and Li G R (2013) BKCa and hEag1 channels regulate cell proliferation and differentiation in human bone marrow-derived mesenchymal stem cells. J Cell Physiol doi:10.1002/jcp.24435
2. Zhou J, Xu C, Wu G, Cao X, Zhang L, Zhai Z, Zheng Z, Chen X, Wang Y (2011) In vitro generation of osteochondral differentiation of human marrow mesenchymal stem cells in novel collagen-hydroxyapatite layered scaffolds. Acta Biomater 7:3999-4006
3. Young R G, Butler D L, Weber W, Caplan A I, Gordon S L, Fink D J (1998) Use of mesenchymal stem cells in collagen matrix for Achilles tendon repair. J Orthop Res 16:406-413
4. Hung M J, Wen M C, Huang Y T, Chen G D, Chou M M, Yang V C (2013) Fascia tissue engineering with human adipose-derived stem cells in a murine model: Implications for pelvic floor reconstruction. J Formos Med Assoc doi:pii: S0929-6646(13) 167-168.
5. Caballero M, Skancke M D, Halevi A E, Pegna G, Pappa A K, Krochmal D J, Morse J, van Aalst J A (2013). Effects of connective tissue growth factor on the regulation of elastogenesis in human umbilical cord-derived mesenchymal stem cells. Ann Plast Surg 70, 568-573
6. Anzalone R, Lo Iacono M, Loria T, Di Stefano A, Giannuzzi P, Farina F, La Rocca G (2011) Wharton's jelly mesenchymal stem cells as candidates for beta cells regeneration: extending the differentiative and immunomodulatory benefits of adult mesenchymal stem cells for the treatment of type 1 diabetes. Stem Cell Rev 7:342-363
7. Chen Q Q, Yan L, Wang C Z, Wang W H, Shi H, Su B B, Zeng Q H, Du H T, Wan J (2013) Mesenchymal stem cells alleviate TNBS-induced colitis by modulating inflammatory and autoimmune responses. World J Gastroenterol 19:4702-4717
8. Wada N, Gronthos S, Bartold P M (2013) Immunomodulatory effects of stem cells. Periodontol 63:198-216
9. Takehara N. Cell therapy for cardiovascular regeneration. (2013) Ann Vasc Dis 6:137-144
10. Ruzzini L, Longo U G, Rizzello G, Denaro V (2012) Stem cells and tendinopathy: state of the art from the basic science to clinic application. Muscles Ligaments Tendons J 2:235-238.
11. Stewart A A, Barrett J G, Byron C R, Yates A C, Durgam S S, Evans R B, Stewart M C (2009) Comparison of equine tendon-, muscle- and bone marrow-derived cells cultured on tendon matrix. Am J Vet Res 70:750-757
12. Izadpanah R, Trygg C, Patel B, Kriedt C, Dufour J, Gimble J M, Bunnell B A (2006) Biologic properties of mesenchymal stem cells derived from bone marrow and adipose tissue. J Cell Biochem 99:1285-1297
13. Yoshimura H, Muneta T, Nimura A, Yokoyama A, Koga H, Sekiya I (2007) Comparison of rat mesenchymal stem cells derived from bone marrow, synovioum, periosteum, adipose tissue and muscle. Cell Tissue Res 27:449-462
14. Harris D T (2013) Umbilical Cord Tissue Mesenchymal Stem Cells: Characterization and Clinical Applications. Curr Stem Cell Res Ther 8:394-399
15. Ryu Y J, Seol H S, Cho T J, Kwon T J, Jang S J, Cho J (2013) Comparison of the ultrastructural and immunophenotypic characteristics of human umbilical cord-derived mesenchymal stromal cells and in situ cells in Wharton's jelly. Ultrastruct Pathol 37:196-203
16. Kadar K, Kiraly M, Porcsalmy B, Molnar B, Racz G Z, Blazsek J, Kallo K, Szabo E L, Gera I, Gerber G, Varga G (2009) Differentiation potential of stem cells from human dental origin—promise for tissue engineering. J Physiol Pharmacol 60 Suppl:167-175.
17. Hsieh J Y, Fu Y S, Chang S J, Tsuang Y H, Wang H W (2010) Functional module analysis reveals differential osteogenic and stemness potentials in human mesenchymal stem cells from bone marrow and Wharton's jelly of umbilical cord. Stem Cells Dev 19:1895-1910.
18. Lovati A B, Corradetti B, Lange Consiglio A, Recordati C, Bonacina E, Bizzaro D, Cremonesi F (2011) Comparison of equine bone marrow-, umbilical cord matrix and amniotic fluid-derived progenitor cells. Vet Res Commun 35:103-121
19. Liras A (2010) Future research and therapeutic applications of human stem cells: general, regulatory, and bioethical aspects. J Transl Med 8:131.
20. Warton T (1656) Adenographia: sive glandularum totius corporis descriptio. London: Wharton. pp. 243-244.
21. Weiss M L, Troyer D L (2006) Stem cells in the umbilical cord. Stem Cell Rev 2:155-162.
22. Gotherstrom C, West A, Liden J, Uzunel M, Lahesmaa R, (2005) Difference in gene expression between human fetal liver and adult bone marrow mesenchymal stem cells. Haematologica 90:1017-1026
23. Karahuseyinoglu S, Kocaefe C, Balci D, Erdemli E, Can A (2008) Functional structure of adipocytes differentiated from human umbilical cord stroma-derived stem cells. Stem Cells 26: 682-691
24. Sarugaser R, Lickorish D, Baksh D, Hosseini M M, Davies J E (2005) Human umbilical cord perivascular (HUCPV) cells: a source of mesenchymal progenitors. Stem Cells 23:220-229
25. Kita K, Gauglitz G G, Phan T T, Herndon D N, Jeschke M G (2010) Isolation and characterization of mesenchymal stem cells from the sub-amniotic human umbilical cord lining membrane. Stem Cells Dev 19:491-502
26. Cooper K, SenMajumdar A, Viswanathan C. (2010) Derivation, Expansion and Characterization of Clinical Grade Mesenchymal Stem Cells from Umbilical Cord Matrix Using Cord Blood Serum. International Journal of Stem Cells 3:119-128
27. Prasanna S J, V S Jahnavi (2011) Wharton's Jelly Mesenchymal stem cells as off-the-shelf cellular therapeutics: A closer look into their regenerative and immunomodulatory properties The Open Tissue Engineering and Regenerative Medicine Journal 4:28-38
28. Can A, Karahuseyinoglu S (2007) Concise review: human umbilical cord stroma with regard to the source of fetus-derived stem cells. Stem Cells. 11:2886-2895
29. McElreavey K D, Irvine A I, Ennis K T, McLean W H (1991) Isolation, culture and characterisation of fibroblast-like cells derived from the Wharton's jelly portion of human umbilical cord. Biochem Soc Trans 19:29S 30. Baudin B, Bruneel A, Bosselut N, Vaubourdolle M (2007) A protocol for isolation and culture of human umbilical vein endothelial cells. Nat Protoc 2:481-485
31. Iacono E, Brunori L, Pirrone A, Pagliaro P P, Ricci F, Tazzari P L, Merlo B (2012) Isolation, characterization and differentiation of mesenchymal stem cells from amniotic fluid, umbilical cord blood and Wharton's jelly in the horse. Reproduction 143:455-468
32. Cardoso T C, Ferrari H F, Garcia A F, Novais J B, Silva-Frade C, Ferrarezi M C, Andrade A L, Gameiro R. (2012) Isolation and characterization of Wharton's jelly-derived multipotent mesenchymal stromal cells obtained from bovine umbilical cord and maintained in a defined serum-free three-dimensional system BMC Biotechnology 12:18
33. Weiss M L, Medicetty S, Bledsoe A R, Rachakatla R S, Choi M, Merchav S, Luo Y, Rao M S, Velagaleti G, Troyer D (2006) Human umbilical cord matrix stem cells: preliminary characterization and effect of transplantation in a rodent model of Parkinson's disease. Stem Cells 24:781-792
34. Von Bahr L, Sundberg B, Loennies L, Sander B, Karbach H, Haegglund H, Ljungman P. GUstafsson B, Karlsson H, Le Blanc K, Ringden O. (2012) Long-term complications, immunologic effects, and role of passage for outcome in mesenchymal stromal cell therapy. Biol Blood Marrrow Transplant; 18(4):557-64
35. European Medicines Agency, Reflection paper on stem cell-based medicinal products. 14 Jan. 2011.
36. Chan R K, Zamora D O, Wrice N L, Baer D G, Renz E M, Christy R J, Natesan S. (2012). Development of a vascularized skin construct using adipose-derived stem cells from debrided burned skin. Stem Cells Int [Epub 2012]
37. McClure, Peters R, Wolf M J, van den Broek M, Nuvolone M, Dannenmann S, Stieger B, Rapold R, Konrad D, Rubin A, Bertino J R, Aguzzi A, Heikenwalder M, Knuth A K (2010) Efficient generation of multipotent mesenchymal stem cells from umbilical cord blood in stroma-free liquid culture. PLoS One 5 doi: 10.1371/journal.pone.0015689.
38. Campard D, Lysy P A, Najami M, and Sokal E M. (2008) Native umbilical cord matrix stem cells express hepatic markers and differentiate into hepatocyte-like cells. Gastroenterology, 134(3):833-48.
39. Ilmer, Matthias, Jody Vykoukal, Alejandro Recio Boiles, Michael Coleman, and Eckhard Alt. (2014) “Two sides of the same coin: stem cells in cancer and regenerative medicine.” The FASEB Journal 28:2748-2761
40. Alt E, Senst C, Murthy S N, Slakey D P, Dupin C L, Chaffin A E, Kadowitz P J, Izadpanah R.(2012) Aging alters tissue resident mesenchymal stem cell properties. Stem Cell Research; 8 (2): 215-225.

1. A method for matched transfer of umbilical cord tissue-derived cells from a donor to a recipient, comprising the steps of:
collecting a section of umbilical cord from a newborn donor;
extracting nucleated tissue from the umbilical cord to derive a stem cell population therefrom; and
administering stem cells from the derived population to a recipient having HLA markers matching the HLA markers of the donor for treating a disease or disorder of the recipient.

2. The method of claim 1, wherein the donor and the recipient are the same.

3. The method of claim 2, wherein the steps of the method are performed in a single medical procedure.

4. The method of claim 3, wherein the steps of the method are performed at the site of delivery of the donor as a point of care site.

5. The method of claim 1, wherein the transfer is allogeneic, and including:
extracting and analyzing a nucleated cell sample of the donor to predict the HLA type of the donor cells upon their maturation, and
preserving the umbilical cord tissue-derived stem cell population for subsequent administration to the recipient having HLA markers matching those of the donor.

6. The method of claim 1, wherein the transfer is allogeneic, and including:
extracting and analyzing a sample of cells derived from umbilical cord blood of the donor to determine therefrom the HLA type of the donor umbilical cord tissue derived-cells upon their maturation, and
preserving the umbilical cord tissue-derived stem cell population for subsequent

7. The method of claim 6, including:
cryopreserving the umbilical cord tissue-derived cell population for storage in a cell bank according to the HLA type of the stored cells to distinguish from stored cells of a different HLA type in the bank.

8. The method of claim 7, including:
cataloging the stored cells of different HLA types in the bank for rapid retrieval of stored cells of a particular HLA type to be administered for allogeneic transfer to a matching recipient.

9. The method of claim 8, including:
posting the catalog of stored cells by different HLA type in the bank on a remotely accessible electronic database for retrieval of matching available donor cells.

10. The method of claim 7, including:
cryopreserving stem cells derived from blood of the umbilical cord of the donor for storage together with the umbilical cord tissue-derived stem cells of the same donor in the cell bank to enhance identification of the HLA type of the stored tissue-derived cells of the donor.

11. The method of claim 1, wherein the stem cells are injected into a growing matched allogeneic fetus via the umbilical cord artery or into the circulation of the growing fetus, to correct genetic diseases of the growing fetus.

12. The method of claim 11, wherein the stem cells are administered for amelioration or correction of mental diseases that are based on a genetically defective background.

13. A method for transferring umbilical cord tissue-derived cells from a donor to a recipient, comprising the steps of:
collecting a section of umbilical cord from a newborn donor;
separating and retaining umbilical cord blood from the umbilical cord;
extracting nucleated tissue from the umbilical cord to derive a stem cell population therefrom;
and combining the retained umbilical cord blood and the stem cells from the same donor are combined and transplanted to a recipient to thereafter attain an enhanced benefit in the recipient owing to the complementary nature of both types of cells.

14. A method for transferring umbilical cord tissue-derived cells from a donor to a recipient, comprising the steps of:
collecting a section of umbilical cord from a newborn donor and obtaining umbilical cord tissue-derived stem cells therefrom;
culturing the umbilical cord tissue-derived stem cells;
collecting media or exosomes from the cultured umbilical cord tissue-derived stem cells, and
administering the recovered media or exosomes to a non-matched allogeneic recipient.

15. A system for processing umbilical cord tissue from a newborn donor at site of delivery of the donor comprising:
a tissue processing unit for processing the umbilical cord tissue in close proximity to the delivery site; wherein the processing is carried out by introduction of a reagent into said unit; and
a receptacle for recovery of a cell preparation from processing of the umbilical cord tissue in the presence of the reagent such that the recovered cell preparation contains regenerative cells in a sufficient number to be used for therapeutic purposes without expansion.

16. The system of claim 15, wherein the reagent comprises a mixture of one or more of a collagenase and a protease.

17. The system of claim 15, including means for retaining the recovered cell preparation for administration to the donor.

18. The system of claim 17, wherein the retaining means retains a number of regenerative cells in the recovered cell preparation sufficient to alleviate conditions associated with pregnancy or delivery related complications suffered by the donor.

19. The system of claim 14, wherein the complications include any among cerebral hypoxia, cerebral palsy, and low APGAR scores.

20. The system of claim 15 further including means for dividing the recovered cell preparation into a plurality of aliquots, for administration of a first aliquot to the donor within the first 24 hours of delivery and subsequent administration of a second aliquot to the donor.

21. The system of claim 17 wherein the retaining means is configured for administration by one of intravenously, intra-arterially, intrathecally, infusion, direct injection, and to a mucosal membrane.

22. A system for matched transfer of umbilical cord tissue-derived cells from a donor to a recipient comprising:
a collector of a sample of human leukocyte antigen expressing cells from a potential donor; and
an analyzer of the sample to predict the human leukocyte antigen expression pattern of the umbilical cord tissue derived cells to be assumed after transfer to a recipient.

23. The system of claim 22 wherein the analyzer uses the results of the sample analysis to confirm that the potential donor is a match to the recipient's HLA.
